(19) United States
(12) Patent Application Publication
(10) Pub. No.: US 2004/0005710 A1
(43) Pub. Date: Jan. 8, 2004

(54) Method for producing recombinant viruses using site-specific recombination

(76) Inventors: Ho-Sun Son, Seoul (KR); Neon-C Jung, Seoul (KR); Eun-Wook Choi, Seoul (KR); Dong-Seung Seen, Seoul (KR); Kyung-Jin Kim, Seoul (KR); Sung-Eun Cho, Gyeonggi-do (KR)

Correspondence Address: Stephen M. De Klerk, BLAKELY, SOKOLOFF, TAYLOR & ZAFMAN LLP, Seventh Floor, 12400 Wilshire Boulevard, Los Angeles, CA, 90025, US

(21) Appl. No.: 10/383,846

(22) Filed: Mar. 7, 2003

(30) Foreign Application Priority Data
Mar. 9, 2002 (KR) 2002-12633

(51) Int. Cl. C12N 15/86; C12N 7/00

(52) U.S. Cl. 435/456; 435/235.1

(57) ABSTRACT

Disclosed are the methods for producing recombinant viruses using in vitro site-specific recombination. In the present invention, linear viral genomic DNAs are digested to two segments, and are subjected to in vitro site-specific recombination with desired genomic materials flanked with site-specific recombinase sites. According to the present invention, since the site-specific recombination mixture can be applied to host cells without further procedures of selecting the recombinant viral genomic DNAs, it is possible to obtain numerous recombinant viruses rapidly at the same time. Thus, the present invention can be used as a high throughput system to generate hundreds or thousands of recombinant viruses.

### BACKGROUND OF THE INVENTION

1. Field of the Invention

This invention relates to a method for producing recombinant viruses and vectors for the same. Furthermore, this invention relates to DNA constructs for the method and the recombinant viruses produced by the method.

2. Description of the Related Art

A recombinant viral vector means a genetically engineered vector, which is used in, for example, the preparation of a vaccine; analysis of function of a gene, a group of genes, or a genomic domain; production of proteins; and gene therapy. Genomic materials, which could be integrated into a viral vector, include any genomic materials such as a gene, cDNA, genomic DNA, DNA sequences encoding peptides or proteins, RNA, anti-sense RNA, siRNA, DNA for si RNA, promoter, enhancer etc. A virus, which is used in the preparation of a vector, includes adenovirus, adeno-associated virus (AAV), retrovirus, Herpes virus and Vacxnia virus etc.

Retrovirus is known as one of the most widely used viruses in gene therapy [see Clive Patience et al. J Virol, 72:2671-2676, 1998, Marshall, Science, 269: 1050-1055,1995]. Retroviral genome has two LTRs, capsid sequences and 3 (three) coding regions (gag, pol and env), and the method of its preparation and its use in vitro and in vivo has been already disclosed [WO9908692A1 and EP 453242]. The clinical application of retroviral vector, however, has the following disadvantages as follows: it forms a replication-competent retrovirus (RCR) (Sharon K. Powell et al. J.Virol., 73:8813-8816, 1999); it has relatively lower gene expression level, and the level decreases continuously in vivo; and the titer of virus is low. Furthermore, the retrovirus cannot transfer a gene to a non-dividing cell [Jaffe, E. M. Cancer Res., 53:2221-2226, 1993; Bender, M. A. et al. J. Virol., 61:1639-1646, 1987].

Adenovirus has a linear genome (sized about 36 kb) and includes replication origin and capsid signals near to the left inverse terminal region (ITR) (103 bp) [Shenk, Adenoviridae: The Viruses and Their Replication. In: Fields B N, Knipe D M Howley P M, ed. Virology. Philadelphia: Raven publishers, 1996: 2111-2148]. Usually, adenoviral vectors have been made by deleting El site of the viral genome, which is essential for the viral replication. The viruses, which include foreign genomic materials substituting El site, are transferred to the packaging cells that provide E1 proteins. Thereby, the viral replication occurs only in the packaging cells. Representative packaging cells include, for example, HEK293 cell, 911 cell, and PER. C6 cell, etc. [Hitt MM et al., Advances in Pharmacology, vol. 40, 137-206 (1997), Wang Y. and Huang S., Drug]. Adenoviral vector has advantages such as safty, affinity to various cells, possibility of infecting dividing cells and higher titer (10¹¹ pfu/ml). Thus, adenovirus has been used in the preparation of a vector expressing heterogeneous genes.

Adeno-associated virus (sized about 4700 bp) has ITRs (sized about 145 bp) as replication origins at each terminus. It can be integrated into the genomic DNAs of various types of host cells safely and specifically.

In order to use a recombinant virus as a vector, it should be modified not to replicate themselves in the infected cells. Therefore, defective viruses, which have deletions of some essential regions of genome for viral replication, are usually employed in the preparation of the recombinant viral vectors. In case of a retrovirus, for example, gag, pol and/or env genes are deleted, and the regions are replaced with desired genomic materials [Bender et al., J. Virol. 61 (1987) 1639]. Regarding an adenovirus, E1, E2 and/or E4 sites are deleted for the preparation of a viral vector [Levrero et al., Gene 101 (1991) 195; Gosh-Choudhury et al., Gene 50 (1986) 161, Van der Vliet et al., (1975)]. On the other hand, a pseudo-virus vector, which consists of only the essential regions (such as, ITR and capsid sequences) of the replication, has been used also for obtaining recombinant viruses (see WO95/02697).

More specifically, conventional methods for the preparation of adenoviruses could be grouped into 3 categories as follows: In the first group, homologous recombination between carrier vectors and adenoviral genomic DNAs is provided, wherein the vectors carry desired foreign genomic materials [Hitt MM et al., Advances in Pharmacology, vol. 40, 137-206 (1997)]. In this method, desired foreign genomic materials are cloned into carrier vectors and then introduced to the packaging cells together with adenoviral vector genomic DNAs in order to induce homologous recombination. A carrier vector usually includes a cassette, such as a promoter, restriction enzyme target sites for gene cloning and poly(A) attachment signals, for the expression of foreign genomic materials. Furthermore, the carrier vector has nucleic acid sequences inducing homologous recombination. In general, a defective adenoviral genome, which lacks LITR, packaging signals, or E1 site, is used in this method. On the other hand, Frank Graham et al. designed a plasmid type vector, which can replicate in E. coil, in order to resolve the problem of the said method of requiring a further step for selecting defective viral genomes from viruses [Bett AJ et al., Proc. Natl. Acad. Sci USA,, vol 91, 8802-8806 (1994)]. The Graham's method, however, also has to use the homologous recombination between carrier vectors and defective adenoviral genomic DNAs, and the lower rate of homologous recombination in packaging cells also made this method inefficient.

The second group includes direct cloning of desired foreign genomic materials to a certain region of adenoviral genome using restriction enzyme and ligase. This method resolved the difficulties caused by the structural complexity of the adenoviral genome (sized about 30 kb). In this method, expression cassette for the desired gene is, firstly, cloned to a carrier vector, which in turn is cloned again into the viral genome using specific restriction enzyme sites within both of the cassette and the viral genome. Next, the cloned cassette and the viral genome are introduced into E. coli, and the desired clones are selected. Afterward, the selected clones are introduced to the packaging cells. Then, the recombinant viruses could be obtained from the packaging cells [Mizuguchi H. and Kay MA., Human Gene Therapy, vol. 10, 2013-2017 (1999)]. In another method of this group, viral genomic DNAs having pTP (preterminal protein) at the region of ITR terminus are extracted from viruses, not from E. coli, in order to increase the productivity of desired recombinant viruses, which in turn are transferred to cell lines after being ligated with the desired genome expression cassette [Okada T. et al., Nucleic Acids Research, vol. 26, 1947-1950 (1998)].

In the third group, a method employs E. coli, which has relatively higher efficiency of homologous recombination, in order to improve the lower recombination rate in the packaging cells [Chartier C. et al., Journal of Virology, vol. 70, 4805-4810 (1996), He T. et al., Proc. Natl. Acad. Sci USA, Vol 95, 2509-2514 (1998)]. According to this method, although the desired genomic materials should be also cloned into carrier vectors, it is possible to obtain relatively higher rate of homologous recombination in the particular E. coli by infecting the cell together with plasmid-type adenoviral genome. Thereby, the desired recombinant viruses could be obtained with higher productivity in comparison to the methods mentioned above. In this method, however, the desired genomic materials should be cloned to carrier vectors. And, a particular E. coli cell line (for example, BJ5183) should be used. Furthermore, the recombinant DNAs expressed in E. coli should be screened, and the screened recombinant DNAs should be transferred to another E. coli cell line (for example, DH5 α) due to the unstableness of the plasmid in the E. coli used for the recombination. Thus, this method is considered as being very complex and not effective.

As disclosed above, the conventional methods for the preparation of recombinant viruses have disadvantages as follows: i) Desired genomic materials should be cloned to carrier vectors; ii) Since the gene cloning of the desired genomic materials should be carried out in a cell, it requires complex and inefficient cloning procedures; iii) The desired recombinant viruses should be selected from host cells before the mass production. Therefore, according to those methods, it takes much time to obtain recombinant viruses, while the productivity is relatively low. Especially, the disadvantages of ii) and iii) have been regarded as the most important problems to be overcome in the preparation of recombinant viruses, thus there have been continuous efforts to resolve these problems so far. On the other hand, according to the development of applications of recombinant viruses, the requirements for more efficient and rapid method for preparing recombinant viruses are increasing.

Thus, we have carried out studies to produce recombinant viruses having desired genes, and we have completed this invention by providing recombinant viral vectors using in vitro site-specific recombination.

### SUMMARY OF THE INVENTION

Any publications referenced herein are hereby incorporated by reference in this application in order to more fully describe the state of the art to which the present invention pertains.

It is important to understand the present invention to note that all technical and scientific terms used herein, unless otherwise defined, are intended to have the same meaning as commonly understood by one of ordinary skill in the art. The techniques used herein are also those that are known to one of ordinary skill in the art, unless stated otherwise.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of suggested method, material or composition is directed.

It is an object of this invention to provide more rapid and more efficient method for producing recombinant viruses.

In order to accomplish this invention, the present invention employs recombinant viral vectors, which are prepared by in vitro site-specific recombination. More preferably, the viruses used in this invention have linear genomic DNAs.

Therefore, it is another object of the present invention to provide recombinant viral vectors using in vitro site-specific recombination.

In the present invention, a vector comprising viral genomic DNAs is cleaved to two or more segments before being applied to in vitro site-specific recombination, in which both of the segments are essential for the viral replication. A DNA fragment including desired genomic materials is inserted between the two genomic DNA segments specifically. Thus, since un-bound viral genomic DNA segments cannot be expressed by themselves, only the viruses having desired DNA fragment shall be expressed. Therefore, one can obtain desired recombinant viruses from host cells directly without further procedures like selecting the desired recombinant viruses from intermediate host cells.

Referring to the site-specific recombination, it is an event occurring naturally in various living organisms. Enzymes for the site-specific recombination contain not only endonuclease activity but also ligase activity, so they recognize a certain part of DNA sequences and replace it with any other corresponding DNA sequences [Yang W. and Mizuuchi K., Structure, 1997, Vol. 5, 1401-1406(9)]. Int/att system from bacterio λ phage, Cre/LoxP system from P1 bacterio phage and FLP-FRT system from yeast are well developed site-specific recombination systems.

Site-specific recombination system has been used in i) expression control of desired genomic materials cloned into recombinant viruses, ii) excising of helper-virus when preparing helper-viral dependent viruses, iii) increasing the efficiency of homologous recombination in the packaging cells, or iv) inducing site-specific recombination between carrier vectors and viral genome in the packaging cells to prepare recombinant viruses [Philip NG et al., Biotechniques, vol. 29. 524-528 (2000), Philip NG et al., Human Gene therapy, vol. 10., 2667-2672 (1999), Hardy S et al., Journal of Viology, vol. 71., 1842-1849 (1997), Parks RJ et al., Proc. Natl. Acad. Sci USA,, vol 93., 13565-13570 (1996)]. However, the said applications of the site-specific recombination disclosed in the prior arts were merely indirect and supplementary uses in vivo or in a cell line, not in vitro.

Thus, there have been no applications of in vitro site-specific recombination for specific insertion of desired genomic materials into linear viral genomic DNAs in order to obtain recombinant viral vectors. In particular, there have been no applications of using viral genomic DNAs, which are separated into two segments.

Therefore, the present invention is characterized by employing in vitro site-specific recombination in the preparation of recombinant viral vectors. It has unexpected advantages in comparison to the prior arts as follows: i) it is possible to obtain recombinant viral vectors more easily and more rapidly by employing in vitro site-specific recombinant; ii) it does not require any further procedure, such as plaque assay, for selecting desired recombinant viruses from intermediate host cells by digesting viral genomic DNAs to two segments before applying them to site-specific recombination; and iii) it is possible to apply the reaction mixture including recombinant viral vectors to host cells without further processes.

Furthermore, according to this invention, homologous recombination in the packaging cells is not required. Nor, particular cell lines for inducing homologous recombination in E. coli are required. FIG. 5 shows the results of comparisons between the method of this invention for the preparation of adenovirus (AdHT System) and the method of the prior art (AdEasy system) in view of processes and time-period. From the results, it is clear that the desired recombinant viruses can be obtained even more rapidly by using the method of this invention.

Furthermore, according to the present invention, since numerous kinds of desired genomic materials are integrated into the sites of viral genomic DNAs accurately in vitro, various genomic materials can be expressed from the same kind of virues in multi-well (e.g. 96-well and 384-well) at the same time. Likewise, since numerous kinds of viral genomes from various types of viruses (such as, adenovirus, retrovirus, adeno-associated virus, bacteriophage, herpes simplex virus, CMV (cytomegalovirus), EBV (Epstein-Barr virus) or VZV (Varicella-zoster virus), etc) are employed, the same kind of genomic material can be expressed from various types of viruses by using an universal common vector (pMaster Vector) containing the desired genomic material.

Accordingly, the present invention also provides significantly useful tools for studying functional genomics of which demands have been increasing greatly, since the completion of genome project [Wang Y. and Huang S., Drug Discovery Today, vol. 5, 10-16 (2000)].

In one embodiment, this invention provides recombinant adenoviruses of which genomic DNAs are in linear form. As depicted in FIG. 2, genomic DNAs of viruses are firstly cleaved to two segments, wherein the segments respectively are essential for the viral replication and packaging. Then, using the cleaved segments as templates, the desired genomic materials containing site-specific recombination sequences are integrated through site-specific recombination. More specifically, E1 site deleted adenovirus is digested to two segments, and recombination sites are introduced into each of the segments in order to induce in vitro site-specific recombination. Other corresponding site-specific recombination sequences are introduced into the desired genomic materials.

Referring to FIG. 2, the left segment of adenoviral genome includes ITR and packaging signals, and the right segment includes other genes essential for viral replication. Recombination and viral packaging, however, cannot occur solely with only one of the two cleaved segments (since the right segment lacks signals for particle formation and the left segment is too short to form a viral particle). Thus, only the viral genome containing the desired genomic materials, which are integrated by site-specific recombination, can be used as a template for replication in a packaging cell.

On the other hand, methods disclosed below can be used for obtaining two segments of viral genomic DNAs. In the first method, viral genomic DNAs are cleaved to two segments with restriction enzyme, wherein each of the segments is essential for viral packaging and has a site-specific recombination site (see FIG. 2). More specifically, one or more restriction enzyme recognition sites are firstly cloned to viral genomic DNAs (see FIG. 1), and then two or more viral DNA segments can be obtained by restriction enzyme treatment. The viral genomic DNAs including restriction enzyme recognition sites could be obtained as a plasmid from E. coli, or be obtained directly from viruses. The obtained plasmid (pAdHTS) has a construct as depicted in FIG. 1. Next, pAdHTS (SEQ. ID No: 1) is cleaved to three segments by restriction enzyme treatment. Except for the segment not related to site-specific recombination and viral replication, other two segments are employed in the preparation of recombinant viral vectors (see FIG. 2).

In the second method, parent viral formation can be prevented. As disclosed in Example 3, the left segment and the right segment of viral DNAs are generated as plasmids respectively, which in turn are made to linear forms, wherein the two segments have site-specific recombination sites, which are different from each other. Referring to FIG. 3, left-segrnent has ITR and packaging signals, and the right-segment has nucleic acids essential for the viral replication. Each of the segments has replication origins (e.g. pBR322 origin or pUC origin etc.) for the replication of DNAs in E. coli, and at least one selection marker (e.g. Ampr, Kanr, Cmr, etc) to permit detection and/or selection. These segments can be provided from other plasmids, respectively.

The viral genomic DNA segments in this invention can be in linear or in circular form. Preferably, they are in linear form. For example, if linear DNA segments generated by restriction enzyme treatment are integrated into pMaster vectors in vitro, linear formed recombinant viral genome could be obtained as depicted in FIG. 4. Thus, in case of circular plasmid DNAs, they are firstly treated with restriction enzyme, such as PacI, to form linear DNA segments and consequently to obtain linear viral genome (see FIG. 4), and then are introduced into the packaging cells to induce viral replication.

In one embodiment, a promoter is inserted into at a site of viral genomic DNAs in order to induce the expression of desired genomic materials (see FIG. 1 and FIG. 2). The promoter, not operating in a cleaved DNA segment, induces the expression of genomic materials, when the materials are introduced by site-specific recombination. In FIG. 6, green fluorescence proteins (GFP) and Gus genes are not expressed in the group of cells that has not been subjected to site-specific recombination.

A promoter, which can be employed in the present invention, includes CMV (cytomegalovirus) promoter for continuous gene expression, RSV (Respiratory syncytial virus) promoter, SV (simian virus) promoter, a promoter system which induces gene expression only in specific environment, a promoter system containing ERE which operates in response to corresponding hormone, a promoter system operating in response to ecdysone, and a promoter system operating in response to tetracycline, etc. The promoter typically controls the expression, optionally with an operator sequence.

In another embodiment, attR sites within two viral genomic DNA segments react with attL sites from carrier vectors including foreign genomic materials, and form attB and attP sites. More specifically, when the two viral genomic DNA segments are treated with carrier vectors having desired genomic materials in the presence of λ integrase, IHF as a co-factor and Xis, attB sites are generated as main products. Vector DNA segments having attP sites are generated also as by-products in the reaction mixture. Numerous recombination systems from various organisms, for example, integrase/att system from bacteriophage λ, the Cre/loxP system from bacteriophage P1, and the FLP/FRT system from the Saccharomyces cerevisiae 2μ circle plasmid, can be employed.

If the mixtures are transferred directly to suitable host cells, which are expected to express recombinant viruses, only the viral genomic DNAs containing attBl-[desired genomic materials]-attB2 can replicate and form viral particles. The viral DNA segments and the vector DNAs, which do not react with each other, and the vector DNA segments having attP sites do not form viral particles.

Referring to FIG. 6, the disappearance of left segments of viral genomic DNAs suggests that the DNA segments having attR sites are recombined. In FIG. 7, PCR products show that the desired genomic materials are inserted exactly into suitable sites of viral genome. PCR reaction is carried out for both of the samples collected before- and after-recombination reaction using primers (Adeno-F primer and Adeno-R primer) that are able to amplify certain regions of viral genome.

Adeno-F primer: 5′-GGTCTATATA AGCAGAGCTG-3′ (SEQ. ID No. 2)

Adeno-R primer: 5′-GTATGGCTGA TTATGATCAG-3′ (SEQ. ID No. 3)

The analysis of PCR products using DNA sequencing shows that viral genome and desired genomic materials are integrated at the target sites and generated [left segment of viral genome-attB1-desired genomic materials-attB2-right segment of viral genome] (see FIG. 2).

Viral replication and packaging are observed after infecting the packaging cells, such as 293 cells, with the reaction mixture including site-specific recombinant viral vectors. These results indicate that the in vitro mixture of this invention can be applied to host cells directly to produce recombinant viruses without further step of selection. That is, according to this invention, without employing intermediate host cells, such as E. coli, for selecting appropriate genomic DNAs, desired recombinant viruses can be produced rapidly. In FIG. 8, the results of site-specific recombination of AdHTP vectors and pMater clones are presented. The expression efficiency of GFP was still higher in the method of this invention compared to the method of the prior art (Ad-GFP control). The activity assays of Gus proteins indicated that the method of this invention did not have adverse-effects on the activities of the proteins expressed. Thus, the recombinant viruses produced in this invention are suitable for the expression of desired genomic materials from infected host cells.

### BRIEF DESCRIPTION OF THE DRAWINGS

FIG. 1 depicts the construct of pAdHTS vector.

FIG. 2 shows the process of site-specific recombination between pAdHTS vector and pMaster clone in vitro.

FIG. 3 depicts the constructs of pAd-Left and pAd-Right.

FIG. 4 shows the process of site-specific recombination of pAd-Left/pAd-Right and pMaster clone in vitro.

FIG. 5 shows processes and time-period for the production of recombinant adenovirus using AdHTS in comparison to those of the methods of prior arts.

FIG. 6 illustrates agarose gel eletrophoretic analysis showing the results of in vitro site-specific recombination of pAdHTS vector and pMaster clone.

FIG. 7 shows the results of PCR, which indicate site-specific recombination of pAdHTS vectors and pMaster clones.

FIG. 8 shows the results of site-specific recombination of AdHTS vectors and pMaster clones. The desired genomic materials in pMaster clones are transferred to adenoviral genome to express the desired proteins.

FIG. 9 shows the results of PCR indicating the site-specific recombination of AdHTS and pMaster-GFP in 96-well.

FIG. 10 shows the results of site-specific recombination of AdHTS and pMaster-GFP in 96-well. GFP gene inserted into pMaster clone is transferred to adenoviral genome to express GFP

### DESCRIPTION OF THE PREFERRED EMBODIMENTS

Preferred embodiments of this invention are described in the following examples. Other embodiments within the scope of the claims herein will be apparent to those skilled in the art from consideration of the specification or practice of the invention as disclosed herein. It is intended that the specification, together with the examples, be considered exemplary only, with the scope and the spirit of the invention being indicated by the claims which follow the examples. The examples herein are meant to exemplify the various aspects of carrying out the invention and not intended to limit the scope of the invention in any way. The examples do not include detailed descriptions of conventional methods employed, such as in the performance of genomic DNA isolation, PCR, and sequencing procedures. Such methods are well-known to those skilled in the art and are described in numerous publications. In addition, all the publications referred herein are integrated hereto as references.

### EXAMPLES

### Example 1

### Site-Specific Recombination Using pAdHTS

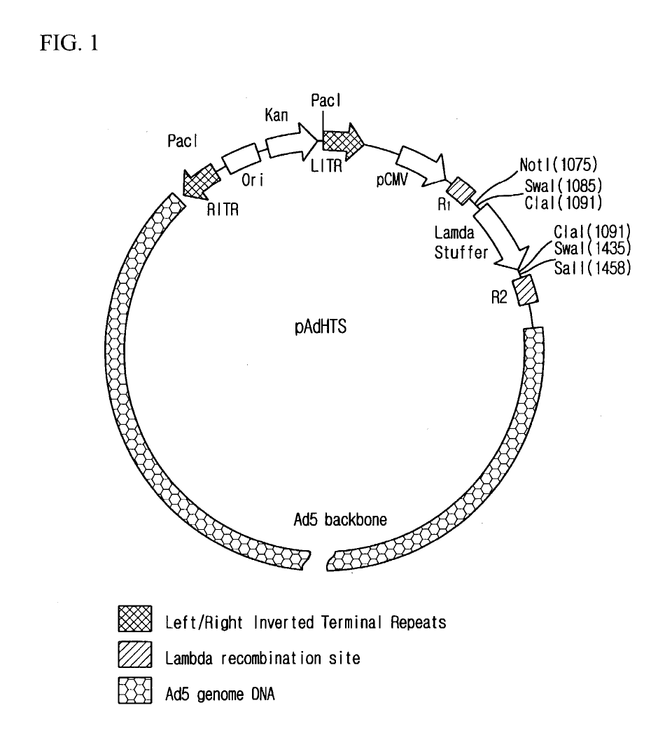

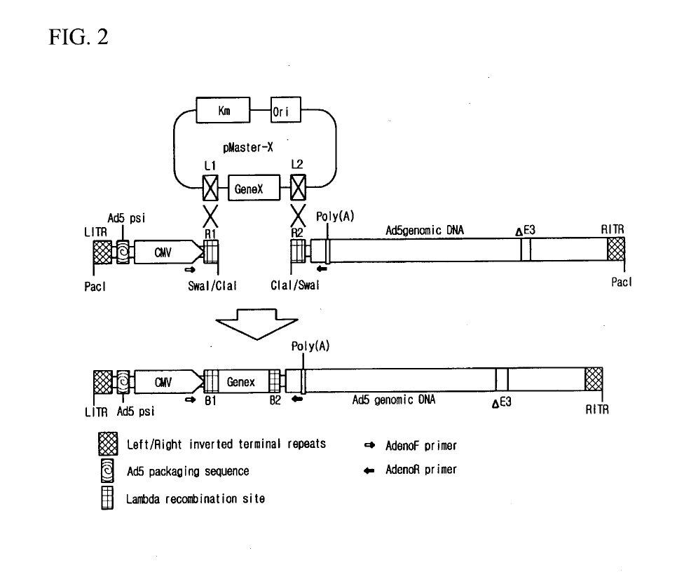

pMaster vector having a desired genomic material flanked with attL sites was prepared (see FIG. 2). Then, pAdHTS (SEQ ID No. 1) having viral genomic DNA flanked with attR sites was prepared using conventional recombination method (see FIG. 1). The pAdHTS vector for this study was generated by deleting E3 site of adenoviral genomic DNAs and by replacing the E1 site with an expression cassette of [CMV promoter]-attRl-[k stuffer DNA]-attR2-[poly A attachment signal], which induced expression of the recombinant desired genomic material (see FIG. 1 and FIG. 2). λ stuffer DNA was prepared to contain restriction enzyme recognition sites, such as SwaI, ClaI or PacI, by amplifying PCR primers having the restriction sites and cloning them thereto. Then, the DNA was treated with SwaI and/or ClaI to generate two DNA segments, wherein each of the segments is essential for the viral replication and packaging. The att sites had the same nucleic acid sequences as those devised by James L. et al.[see Hartley J L. et al., Genome Research, vol. 10, 1788-1795 (2000)]. pMaster vectors flanked with the mutant attL sites (attL1 and attL2) were cloned with GFP cDNA and Gus CDNA, respectively, using recombination methods (see FIG. 2, Gene-X site of pMaster vector). As the result, the plasmid vectors comprising expression cassettes of attLl-[desired genomic materials]-attL2 were generated.

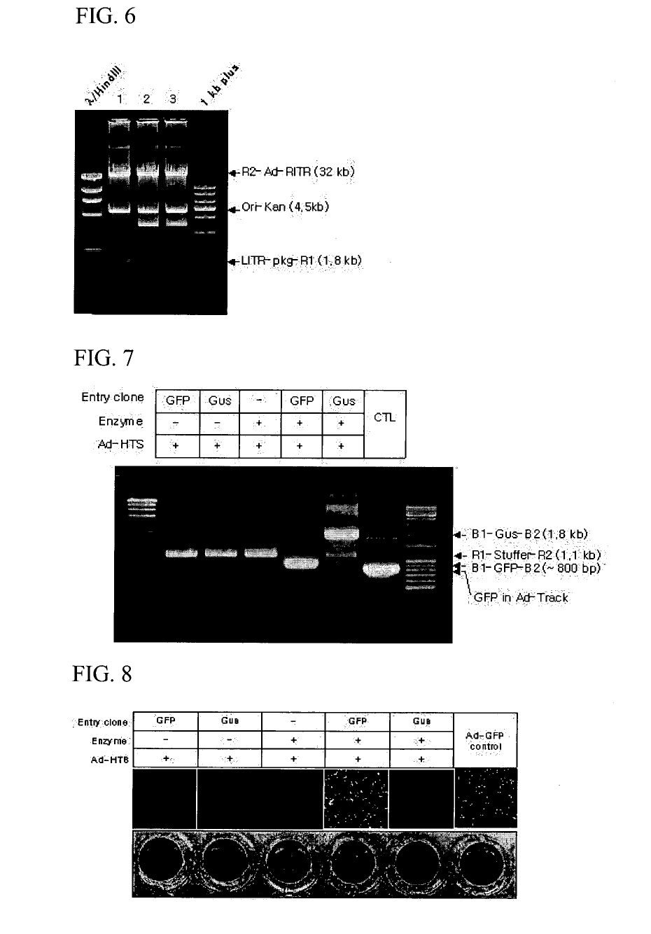

DNAs of pAdHTS (1 μg) were purified using phenol/chloroform after treating them with SwaI and/or ClaI enzyme(s). The pMaster-GFP (100 ng) containing GFP cDNA, pMaster-Gus (100 ng) containing Gus cDNA, integrase (100 ng), IHF (10 ng), and Xis (10 ng) were reacted in a buffer (50 mM Tris, pH 7.8, 16.5 mM NaCl, 35 mM KCl, 5 mM Spermidine, 0.25 mM EDTA, 3% Glycerol) at 25° C. for 1 hr. Then, integrase, IHF and Xis were removed from E. coli according to the common method [Kotewicz M et al., Journal of Biological Chemistry, 1980, vol. 255, 2433-2439; Nash H A. and Robertson C A. Journal of Biological Chemistry, vol. 256, 9246-9253 (1981); Cho E H et al., Journal of Bacteriology, vol. 182, 5807-5812 (2000)]. The results of agarose gel electrophoretic analysis indicated that pAdHTS treated with PacI generated three DNA segments (32 kb, 4.5 kb and 1.8 kb) (FIG. 7 Lane 1). Where site-specific recombination occurred, it was observed that the segment (sized about 1.8 kb) was disappeared (FIG. 7 Lanes 2 and 3). In order to confirm this, PCR was carried out using adeno-F primer and adeno-R primer, thereby the desired sized PCR products (about 32 kb and about 4.5 kb) could be observed where the site-specific recombination was induced (see FIG. 6).

The PCR products were isolated from agarose gel, and DNA sequencing was carried out for the PCR production. As the result, it was determined that the PCR products of Lanes 2, 3 and 4 were λ DNAs resulting from insufficient cleavages of adenoviral genome. And, it was determined that the PCR product of Lane 5 was attB1-GFP-attB2, and that the PCR product of Lane 6 was attB1-Gus-attB2. That is, recombination between two adenoviral genomic segments and one pMaster clone was induced.

### Example 2

### Preparation of Viruses Using pAdHTS

2 micro-liter of the reaction mixture (100 ng of DNA) obtained from Example 1 was introduced to 293 packaging cells. Then, the 293 cells were transferred to 24-well (5×10⁴ cells/well) with DMEM (5% bovine fetal serum). After 24 hrs, recombination induced reaction mixture (2 micro-litter) and LipofecAmine2000 (0.5 micro-litter) (from Invitrogen Inc.) were transferred to the cells in accordance with the manufacturer's protocol. After 7 days from the transfer, CPE was observed under a microscope, which indicated the formation of viruses in the cells. At that moment, the cells were harvested and were centrifugated. The resulting precipitates were suspended in 100 micro-liter of DMEM 10% bovine fetal serum. The cells were treated at −70° C. for 1 hr followed by at 37° C. for 5 min. These freezing and thawing were repeated 3 times. The lysate was removed by centrifuge to produce viral mixture. Afterward, 293 cells were infected with the viral mixture. Plaque assay was carried out to detect the formation of suitable viruses.

For plaque assay, 2×10⁵ cells were transferred to 6-well plate. After 24 hrs from the transfer, 10 micro-liter of the viral mixture mixed with 400 micro-liter of DMEM 400 was plated on the cells for 1 hr in order to induce viral adsorption. Plaques of viruses were assayed for 2 weeks, after solidifying the cells with 3 ml of DMEM to which 1% low melting-point agarose (SeaPlaque Agarose; FMC) was added. The titer of viruses was determined as the number of viral plaques per 1 ml of viral mixture. After 2 weeks, in order to detect product efficiency of the desired recombinant viruses, 500 μg of X-Gluc (5-bromo-4-chloro-3-indolil-beta-D-gluchronic acid) as substrate of Glu protein was added to the solidified agarose. After 24 hrs, the number of blue colored plaques was calculated. The titer of viruses was 106107 PFU per 1 ml. The production efficiency of recombinant viruses was determined by the recombinant viruses expressing Gus genes. All the viruses were observed as blue plaques, which suggested that 100% of the viruses obtained in the present invention were recombinant viruses. That is, only the viral genome, including desired genomic materials, replicated and formed viral particles. This had occurred because even though the efficiency of the site-specific recombination was not 100%, the separated DNA segments themselves alone and the by-products of site-specific recombination did not replicate.

As depicted in FIG. 8, recombinant viruses expressing GFP and Gus, respectively, were prepared as mentioned above. The 293 cells were infected with the recombinant viruses. Then, the expression of desired proteins (GFP or Gus) was detected under the fluorescence microscope (for GFP) or by adding X-Gluc substrate (for Gus). As shown in FIG. 8, only the group of viruses to which site-specific recombinase was added generated recombinant viruses and expressed desired proteins (see FIG. 8 Lanes 4 and 5).

### Example 3

### Preparation of Recombinant Viruses Using pAd-Left and pAd-Right

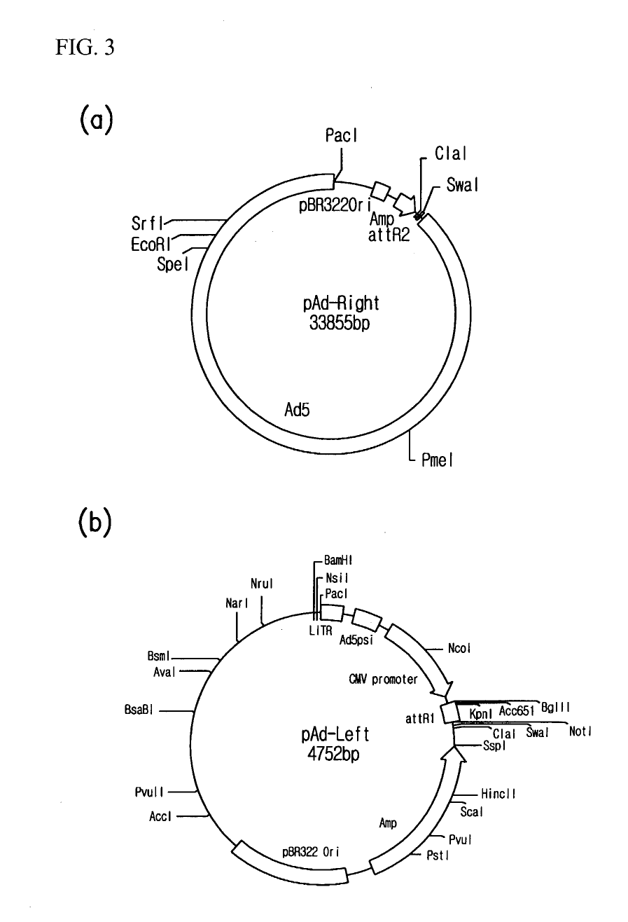

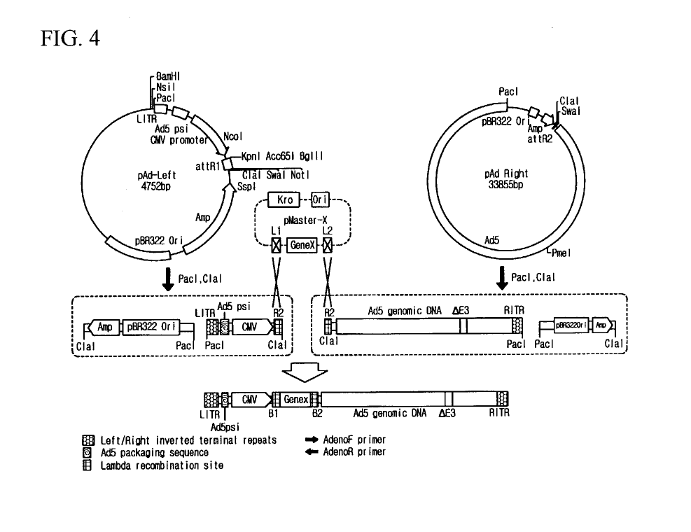

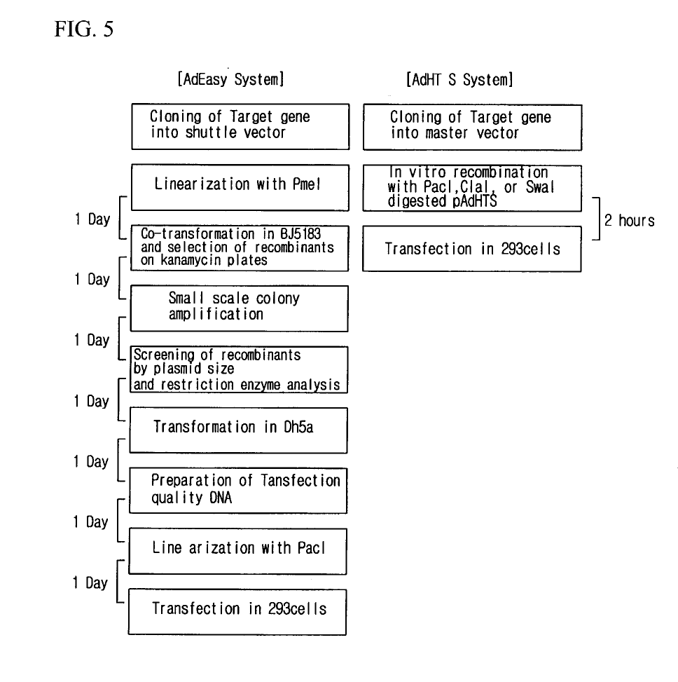

pAd-Left (SEQ. ID No. 4) and pAd-Right (SEQ ID No:5) were prepared (see FIG. 3). Firstly, pAdHTS vector was treated with PacI and/or SwaI. Then, the left fragment containing LITR and packaging signals were ligated with Amp-Ori DNA from PCR amplification in order to form pAd-Left. Likewise, the right fragment (sized about 33 kb) was ligated with Amp-Ori DNA to form pAd-Right. In this study, PCR primer for Amp-Ori included ampicillin resistant gene of pBR322 DNA, a region for amplification of replication origins, and PacI and/or SwaI linker(s) for cloning restriction enzyme recognition sites. The obtained plasmid DNAs of pAd-Left and pAd-Right were generated from DH5a (Qiagen, Midiprep kit), which in turn were digested with SwaI and PacI. Using the respective plasmid DNA, site-specific recombination was induced as disclosed in Examples 1 and 2, so as to produce recombinant viruses. Briefly, linear DNAs of pAd-Left (100 ng of DNA) and linear DNA of pAd-Right (800 ng of DNA) were reacted in the presence of pMaster-GFP having GFP CDNA (100 ng), integrase (100 ng), IHF (10 ng) and Xis (10 ng) within a buffer for 1 hr. The resulting mixture was transferred to 293 packaging cells as disclosed in Example 2. After 7 days, viruses were obtained from cell debris, and plaque assay was carried out.

### Example 4

### Isolation and Manipulation of AdHTS-Terminal Protein Complex

In order to obtain AdHTS viruses, pAdHTS were digested with restriction enzyme of PacI, which in turn were transferred to 293 packaging cells to induce viral packaging. Mass production of AdHTS viruses was carried out by transferring about 5×10⁵ of 293 cells to 30 cell culture dishes (150 mm of diameter) together with DMEM (5% bovine fetal serum). After 24 hrs, AdHTS virus was infected to 5 MOI. After 48 hrs from viral infection, cells were harvested from the dishes with cell scraper, and then were centrifugated. Afterward, the precipitates were suspended in a solution of 10 mM of Tris and 1 mM of MgCl₂. The suspension was treated at −70° C. for 1 hr followed by at 37° C. for 5 min. The freezing and thawing were repeated 3 times. The cell lysate was centrifugated, and the precipitates were removed to obtain viral mixture. In order to obtain infectious viral particles, CsCl gradient ultra-centrifugation was employed. 1.4 g/ml of CsCl solution was poured into a tube (BeckMan), and then 1.2 g/ml of CsCl solution was added to the solution again. After adding 20 ml of viral mixture to the solution while maintaining the concentration gradient, the solution was centrifugated with ultra-centrifuge at 25,000 rpm for 2 hrs, thereby 1×10¹² of infectious viral particles were isolated from defective viral particles and cell debris.

Two different methods were used in order to prepare DNA-terminal protein complex from AdHTS viruses. In the first method, 6 M guanidine HCl was added to the same amount of AdHTS viral solution, and then 1 mg of ethidium bromide and saturated CSCl/4M guanidin HCl solution were added to make the final volume to 5 ml. Next, the solution was ultra-centrifugated at 650,000 g for 4 hrs. Isolated viral DNA-terminal protein complex band was detected with 365 nm of ultraviolet rays, then the DNAs corresponding to the band were extracted. The extracted DNAs were desalted for use in the following studies. In the second method, adenoviral solution, which had a mixture of 6 M guanidine HCl and the same amount of AdHTS viral solution, was transferred to superdex 16/30HR S-200 column (Amersham-Pharrnacia). While developing the solution in 0.4 ml/min of flow rate, optical densities for each of the subsequent 2 ml fractions obtained continuously were detected at 260 nm and 280 nm. AdHTS DNA-terminal protein complex was detected on 1% agarose gel from the collection of peak fractions.

The AdHTS DNA-terminal protein complex obtained from above two methods was digested with SwaI (NEB) and stored at −70° C. before being used in other studies.

### Example 5

### Plaque Assays for Viruses Resulted From Site-Specific Recombination with pMaster-GFP

10 ul of in vitro reaction mixture of pAdHTS vector from Example 2 and pMaster-GFP were transferred to 293 packaging cells as disclosed in Example 2. After 7 days, plaque assay was carried out for the viruses extracted from the cell lysate. In the same manner, plaque assay for pAd-left/pAd-right of Example 3 and for AdHTS-DNA-TPC of Example 4 were carried out, and the results are represented in Table 1 below.

TABLE 1
The results of plaque assays for viruses resulted
from site-specific recombination between each of
adenoviral vectors and pMaster-GFP.

| Mater Clone | pMmaster-GFP | | | |
|---|---|---|---|---|
| | | | AdHTS- | pAd-left |
| adenoviral vector | No recombination | PAdHTS | TPCC | pAd-Right |
| total plaques | 0 | 50 | 74 | 65 |
| (white spot) | | | | |
| GFP-positive | 0 | 50 | 74 | 65 |
| plaques | | | | |

### Example 6

### Multi-Well Production of Recombinant Adenovirus

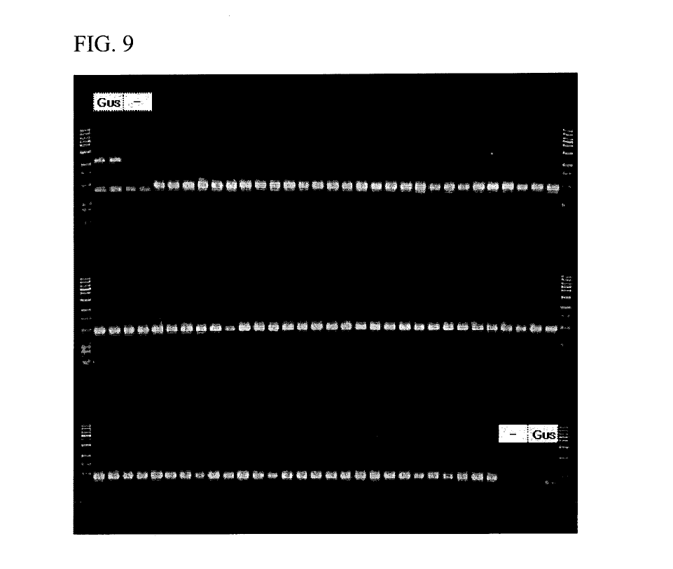

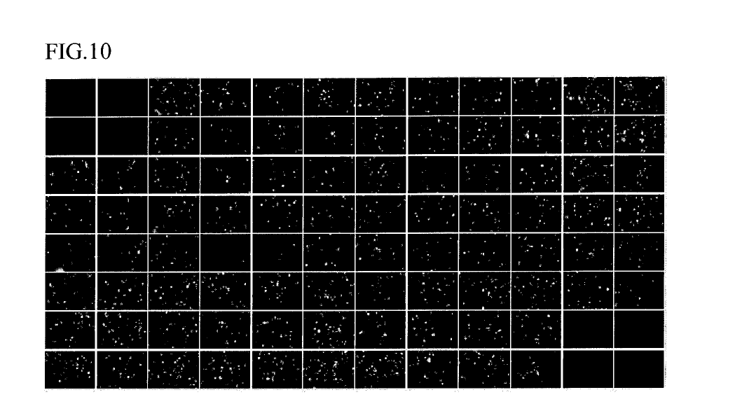

45 ng/well of SwaI treated AdHTS-DNA-terminal protein complex in Example 4 were transferred to each well of 96-well PCR plate, which in turn were subjected to site-specific recombination as in Example 1. Briefly, 100 ng of integrase, 10 ng of IHF, 10 ng of Xis, reaction buffer (50 mM Tris, pH 7.8, 16.5 mM NaCl, 35 mM KCl, 5 mM spermidine, 0.25 mM EDTA, 3% glycerole), and 5 ng of pMaster-GFP including GFP cDNA were transferred to each well. As a control, 5 ng of pMaster-Gus including Gus cDNA was transferred to 4 (four) wells respectively, and reaction was continued for 1 hr. In order to confirm site-specific recombination, PCR reaction was carried out using Adeno-F primer and Adeno-R primer. As the result, PCR products corresponding to GFP were detected in 88 wells except in the controls (see FIG. 9). Similar to Example 2, 10 ul (DNA 50 ng) of the reaction mixture, which was confirmed as including site-specific recombinant, was transferred to 96-well plate, wherein each of wells contained about 5×10³ of 292 packaging cells which were incubated for 24 hrs and wherein each of wells was coated with poly-L-lysin. The cells containing DNAs showed fluorescence. CPE was observed 4-7 days after the transfer, which indicated viral formation. At 10^{th} day from the transfer, 96-well plate was treated at −70° C. for 1 hr followed by at 37° C. for 5 min. The freezing and thawing were repeated 3 times to obtain cell lysate. The cell lysate was centrifugated at 2,500 rpm for 10 min. Then, the 293 cells in 96-well plate were transfected with the supernatant to confirm the formation of viruses expressing GFP (see FIG. 10). In the same manner, the expression efficiency of recombinant viruses was determined for the other adenoviral vectors, such as pAdHTS and pAd-Left/pAd-Right, in 96-well, respectively, and the average efficiency of recombinant viruses is presented on Table 2 below.

TABLE 2
The expression efficieny of recombinant viruses in 96-well

| Master clone | pMaster-GFP | | |
|---|---|---|---|
| adenoviral vector | PAdHTS | pAd-Left/pAd-Right | AdHTS-TPC |
| expression efficiency of | 62.5 | 47.9 | 99.0 |
| recombinant viruses in | | | |
| 96-well | | | |

What is claimed is:

1. A method for preparing linear recombinant viral vectors, wherein desired DNAs are inserted into viral genomic DNAs by in vitro site-specific recombination, which comprises:
i) providing viral genomic DNA segments containing site-specific recombination sequences and base sequences essential to viral replication and packaging,
ii) providing carrier vectors comprising at least one desired DNAs and site-specific recombination sequences; and
iii) reacting the DNA segments of i) and the carrier vectors of ii) in vitro.

2. The method for preparing linear recombinant viral vectors as claimed in claim 1, wherein the viral genomic DNA segments of i) consists of 2 (two) DNA segments which respectively are essential for viral replication and packaging, and include site-specific recombination sequences.

3. The method for preparing linear recombinant viral vectors as claimed in claim 1, wherein the viral genomic DNAs originate from any one of the group consisting of adenovirus, retrovirus, adeno-associated virus, bacteriophage, coronavirus and herpes simplex virus, CMV (cytomegalovirus), EBV (Epstein-Barr virus) and VZV (Varicella-zoster virus).

4. The method for preparing linear recombinant viral vectors as claimed in claim 3, wherein the viral genomic DNAs originate from adenovirus.

5. The method for preparing linear recombinant viral vectors as claimed in claim 4, wherein the adenovirus is characterized by being deleted of E1, E2, E3 and/or E4 sites.

6. The method for preparing linear recombinant viral vectors as claimed in claim 1, wherein the site-specific recombination sequences are att sites.

7. A linear recombinant viral vector prepared according to any one of methods of claims 1 to 6.

8. A method for preparing recombinant viruses having linear genomic DNAs, which comprises the steps of:
i) providing viral genomic DNA segments containing site-specific recombination sequences and base sequences essential to viral replication and packaging;
ii) providing carrier vectors containing at least one desired DNAs and site-specific recombination sequences;
iii) reacting the genomic DNA segments of i) and the carrier vectors of ii) in vitro using site-specific recombinase to produce mixture comprising expressive linear recombinant viral vectors; and
iv) transfecting the mixture of iii) to host cells.

9. The method for preparing recombinant viruses having linear genomic DNAs as claimed in claim 8, wherein the viral genomic DNAs of i) consists of 2 (two) DNA segments which respectively are essential for viral replication and packaging, and include site-specific recombination sequences.

10. The method for preparing recombinant viruses having linear genomic DNAs as claimed in claim 8, wherein the virus is any one of the group consisting of adenovirus, retrovirus, adeno-associated virus, bacteriophage, coronavirus and herpes simplex virus.

11. The method for preparing recombinant viruses having linear genomic DNAs as claimed in claim 10, wherein the virus is adenovirus.

12. The method for preparing recombinant viruses having linear genomic DNAs as claimed in claim 8, wherein the adenovirus is characterized by being deleted of E1, E2, E3 and/or E4 sites.

13. The method for preparing recombinant viruses having linear genomic DNAs as claimed in claim 8, wherein the site-specific recombination sequences are att sites.

14. A recombinant virus having linear genomic DNAs prepared according to any one of methods of claims 8 to 13.
